(19) United States
(12) Patent Application Publication
(10) Pub. No.: US 2006/0116663 A1
(43) Pub. Date: Jun. 1, 2006

(54) Electro-osmotic fluid delivery device and method

(76) Inventors: Ashok V. Joshi, Salt Lake City, UT (US); Sai Bahavaraju, Salt Lake City, UT (US); John H. Gordon, Salt Lake City, UT (US); Lawrence Domenick, Park City, UT (US)

Correspondence Address: CERAMATEC, INC., 2425 SOUTH 900 WEST, SALT LAKE CITY, UT, 84119, US

(21) Appl. No.: 11/173,813

(22) Filed: Jul. 1, 2005

Related U.S. Application Data
(63) Continuation-in-part of application No. 10/908,804, filed on May 26, 2005.
Application No. 10/908,804 is a continuation-in-part of application No. 10/137,661, filed on May 1, 2002.

(51) Int. Cl. A61K 9/22

(52) U.S. Cl. 604/892.1

(57) ABSTRACT

The present invention is directed to a fluid delivery device including a mechanism for decreasing the time required to achieve a constant fluid delivery rate. The fluid delivery device includes an electrochemical cell including a first half-cell and a second half-cell. A controller is operably connected to a first electrode positioned within the first half-cell and a second electrode positioned within the second half-cell. An ion-exchange member is positioned between the first half-cell and the second half-cell. A first reservoir contains a fluid to be delivered and, a displaceable member is positioned between the electrochemical cell and the reservoir, wherein movement of the displaceable member facilitates delivery of the fluid from the reservoir.

### CROSS-REFERENCE TO RELATED APPLICATIONS

This application is a continuation-in-part of U.S. patent application Ser. No. 10/908,804, filed May 26, 2005 which is a continuation in part of U.S. patent application Ser. No. 10/137,661, filed May 1, 2002, and is related to U.S. patent application Ser. No. ______, entitled, “Fluid Delivery Device Having An Electrochemical Pump With An Ion-Exchange Membrane And Associated Method,” filed simultaneously on this date; the contents of these applications are incorporated herein by reference.

### TECHNICAL FIELD

The present invention relates generally to an electro-osmotic fluid delivery device utilized to deliver small volumes of fluid with high precision and accuracy. In particular, the present invention more specifically relates to methodologies to improve the startup characteristics of an electro-osmotic fluid delivery device.

### BACKGROUND OF THE INVENTION

Today's fluid delivery devices utilize various mechanisms of delivery. Such mechanisms include pressure, mechanical, and electrochemical means. Another delivery mechanism includes an electro-osmotic cell coupled with the delivery device to form an electro-osmotic delivery device. The electro-osmotic device operates through the combination of an electrochemical cell and an ion-exchange membrane to create a driving force for fluid delivery.

An electro-osmotic fluid delivery device described in U.S. patent application Ser. No. 10/137,661 utilizes electroosmosis and osmosis to deliver a fluid wherein an electric controller is actuated whereupon an electrical circuit is completed to cause electrode reactions to occur such that water is extracted from the first electrode half-cell and ultimately driven across an ion-exchange membrane into the second electrode half-cell. The water moves a displaceable member which in turn displaces the fluid held in the reservoir. The fluid delivery rate is controlled by the magnitude of current output from the electrical controller.

Generally, two types of osmotic transport are simultaneously occurring with an operating electro-osmotic cell. The primary type of osmosis is electro-osmosis, whereby charged ions—dissociated salts—are driven across an ion-exchange membrane as the cell is operated, thereby dragging water molecules along with them. The second form of transport is osmosis due to environmental conditions. Osmosis is the transfer of a solvent, e.g., water, across a barrier, generally from an area of lesser solute concentration to an area of greater solute concentration.

On starting operation of the electro-osmotic fluid delivery device, the relative concentrations of salts within the half-cells of the electro-osmotic cell change, causing significant changes in the amount of fluid to be delivered. As operation of the device is continued, the passage of ions—salts—across the membrane of the cell causes a steady increase in the salt concentration within the first half-cell and a steady decrease in the second half-cell. The concentration difference will allow the environmental osmosis flux to develop. Ultimately, a steady-state delivery rate is reached due to establishment of steady-state concentrations in both half-cells. At steady-state, environmental osmosis becomes a significant component in the overall flux. The additional solvent transfer causes an increase in the overall fluid amount contained in the second half-cell containing the device product chamber, increasing the rate of fluid delivery.

One observed drawback of today's elctro-osmotic fluid delivery devices involves the delay in achieving the constant delivery rate after the startup of the electro-osmotic cell's operation. Also, as the operation of the device is continued over a period of time, it has been observed that the delivery rate is unreliable and inconsistent, even though the current rate between the first half-cell and the second half-cell is maintained at a constant rate.

The present invention is directed to resolve these and other issues.

### SUMMARY OF THE INVENTION

The present invention is directed to an electro-osmotic delivery device capable of achieving a substantially constant fluid delivery rate in a quicker amount of time relative to today's fluid delivery devices.

The present invention is directed to a fluid delivery device comprising a means for decreasing the time to achieve a desired constant fluid delivery rate. The fluid delivery device further includes an electrochemical cell including a first half-cell and a second half-cell. A controller is operably connected to a first electrode positioned within the first half-cell and a second electrode positioned within the second half-cell. An ion-exchange member is positioned between the first half-cell and the second half-cell. A first reservoir contains a fluid to be delivered and, a displaceable member is positioned between the electrochemical cell and the reservoir, wherein movement of the displaceable member facilitates delivery of the fluid from the reservoir.

A further aspect of the present invention is directed to a method for decreasing the time to achieve a steady-state osmotic transfer in a fluid delivery device. The method includes pre-loading the first half-cell with an electrolyte having a first ionic concentration; and, pre-loading the second half-cell with an electrolyte having a second ionic concentration that is greater than the first ionic concentration of the first half-cell, wherein environmental osmosis between the first half-cell and the second half-cell is quickly established.

Another aspect of the present invention is directed to a method for decreasing the time to achieve a steady-state osmotic transfer in a fluid delivery device having a first and second half-cell. An electrical current is generated between the first half-cell and the second half-cell to achieve a steady-state osmotic transfer between there between. The electrical current is decreased in response to achieving a desired fluid delivery rate such that the fluid delivery rate is maintained constant.

An object of the present invention is to substantially reduce the time taken by a fluid delivery device to reach a constant delivery rate after starting operation of the electro-osmotic cell.

Another object of the present invention is to increase the reliability and consistency of the delivery rate of the device.

These and other objects will become apparent to one of ordinary skill in the art in light of the present specification, claims, and drawings appended hereto.

### BRIEF DESCRIPTION OF THE DRAWINGS

FIG. 1 depicts a cross-sectional side view of the fluid delivery device of the present invention with an internal first half-cell;

FIG. 2 depicts a cross-sectional side view of an alternate embodiment of the present invention having an external first half-cell; and,

FIG. 3 depicts a cross-sectional side view of an alternate embodiment of the present invention having an external first half-cell with the first electrode positioned on the external surface of the device.

### DETAILED DESCRIPTION OF THE INVENTION

While the present invention is capable of embodiment in many different forms, there is shown in the drawings and will herein be described in detail exemplary embodiments of the invention with the understanding that the present disclosure is to be considered as an exemplification of the principles of the invention and is not intended to limit the broad aspect of the invention to the embodiments illustrated.

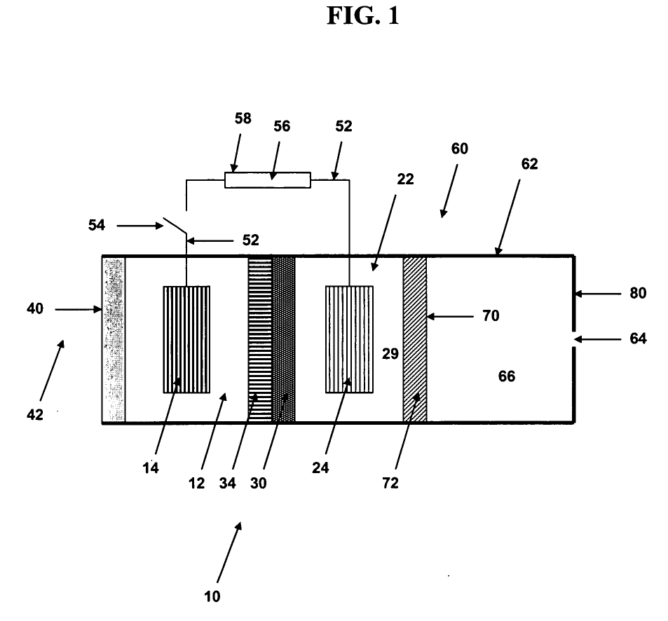

It will be understood that like or analogous elements and/or components, referred to herein, may be identified throughout the drawings with like reference characters. It is also to be understood that the embodiments shown in FIGS. 1-3 are merely a schematic representation of the electro-osmotic delivery device of the present invention. As such, some of the components have been distorted from their actual scale for pictorial activity.

The present invention may be useful as an implantable medical device for delivering a medicament to a patient over a period of time and is generally described herein relative to such an implantable device. Although the present invention is shown in conjunction with implantable devices, it should be noted that the teachings contained within the specification and the appended claims may be translated to other devices and applications without departing from the intended scope of this disclosure.

Shown in FIG. 1 is an embodiment of the present invention wherein an electro-osmotic delivery device 60 comprises an electro-osmotic cell 10, a displaceable member 70, and a reservoir 62. The electro-osmotic cell 10 includes a housing 80, within which a first half-cell 12 and second half-cell 22 are situated. Within the first half-cell 12 and the second half-cell 22 are electrodes with a first electrode 14 in the first half-cell 12 and second electrode 24 in the second half-cell 22. The electro-osmotic cell 10 includes an electrolyte in electrical communication with both the first electrode 14 and the second electrode 24, enabling operation of the cell.

The first 14 and second 24 electrodes preferably comprise an anode and a cathode—and vice versa—and are separated by an ionic-exchange membrane 30 placed there between. Preferably, the ion-exchange membrane 30 is situated within the housing 80 and between the two half-cells 12, 22. Alternatively, the first half-cell 12 need not be positioned inside the device 60 and can be positioned either on the outside wall of the device or entirely away from the housing 80. In such a configuration, the first half-cell 12 is directly exposed to the body fluid 42 and a porous separator 40 can be placed directly adjacent to the ion-exchange membrane 30.

In one embodiment of the present invention, the first electrode 14 of the electro-osmotic cell is constructed from an active metal anode that may include a solid pellet, mesh, or metal powder type electrode fabricated from, for example, zinc, iron, magnesium, aluminum or other corrosion stable metal and alloys. The second electrode 24 is constructed from a conventional current collector that can be readily reduced when coupled with the first electrode 14. The second electrode 24 may be fabricated from porous silver chloride, manganese dioxide, or other materials that can be readily reduced or may catalyze a reduction reaction when coupled with the first electrode, e.g., reduction of oxygen or evolution of gaseous hydrogen from water—when coupled with the active metal anode. The ion-exchange membrane 30 separating the first 14 and second 24 electrodes of this embodiment is a cation exchange membrane. The cationic exchange materials from which the membrane 30 may be made are well known in the art and do not require extensive elaboration. Exemplary materials include perfluorosulfonate membranes known in the art and available under the trade name Nafion® (DuPont). Additional preferred resins are the copolymers of styrene and di-vinyl benzene having sulphonate ion as the charge group which has high selectivity sodium ions. Examples of these membranes include Neosepta type membranes CM-1, CM-2, CMB, C66-F and others, commercially available from AMERIDIA (www.ameridia.com).

In an alternate embodiment of the electro-osmotic cell of the present invention, the configuration of the first 14 and second 24 electrodes are constructed opposite to the embodiment described above. That is, the first electrode 14 is comprised of porous silver chloride, manganese dioxide, or other materials that can be readily reduced or may catalyze a reduction reaction, e.g., reduction of oxygen or evolution of gaseous hydrogen from water-when coupled with the active metal anode. The second electrode 24 is therefore comprised of an active metal anode that can be a solid pellet, mesh, or metal powder type electrode fabricated from, for example, zinc, iron, magnesium, aluminum, or another corrosion stable metal or alloy. The ion-exchange membrane 30 separating the first and second electrodes of the alternate embodiment is an anion exchange membrane. The anionic exchange materials from which the membrane 30 may be made are well known in the art and do not require extensive elaboration. Exemplary materials include polymeric membranes with styrene-divinyl benzene backbone with quaternary ammonia charge groups, known in the art and available under the trade name AFN® (Ameridia).

Either of the above electro-osmotic cell embodiments can be incorporated into an electro-osmotic fluid delivery device of the present invention.

To regulate the operation of either embodiment of the electro-osmotic cell, the cell preferably includes a controller 52 for controlling the electrochemical cell. The controller 52 is connected to the first electrode 14 and the second electrode 24 and comprises an electrical circuit, e.g., an activation switch 54, a control circuitry 56, and a resistor 58. The controller 52 facilitates control of the time course and magnitude of current that flows through the electrodes 14, 24 of the electro-osmotic cell. The controller 52 is also capable of adjusting the delivery rate in various manners and wave forms. Additionally, the controller 52 can aid in fast shutoff of fluid delivery as described in U.S. Patent Application Publication No. US2004/0144646; the contents of which are expressly incorporated herein by reference.

The electrical controller 52 facilitates control of the rate of delivery of fluid out of reservoir 62. The electrical controller 52, in cooperation with the activation switch 54, control circuitry 56, and resistor 58, are operably coupled to the first electrode 14 and second electrode 24 via conventional electrical conduit to control the rate of water transfer from the external source 42 to the second half-cell 22, as well as the starting, stopping, and length of the operation. It is to be understood that the resistor 58 may be replaced by a more sophisticated electrical element(s)—e.g., variable resistance, rheostat—without departing from the present invention.

Generally, electro-osmotic delivery device 60 is associated with a water-rich environment so that water may be allowed into the cell 10 preferably through a protective porous separator 40. The protective porous separator 40 is positioned at an end of the housing 80 proximate the first half-cell 12 and distally from the ion-exchange membrane 30. Thus, the protective porous separator 40 is at least permeable to H₂O and NaCl molecules, and enables water and ions from an external source 42, e.g., an inside of a living being's body, to migrate into the first half-cell 12. The protective porous separator 40 may be fabricated from any of a number of materials, including, but not limited to: metals, glass, porous protective gel, natural and synthetic plastics, and composites. The use of the separator 40 is not required and, accordingly, when not used, the first electrode 14 can be exposed directly to fluid, if desired.

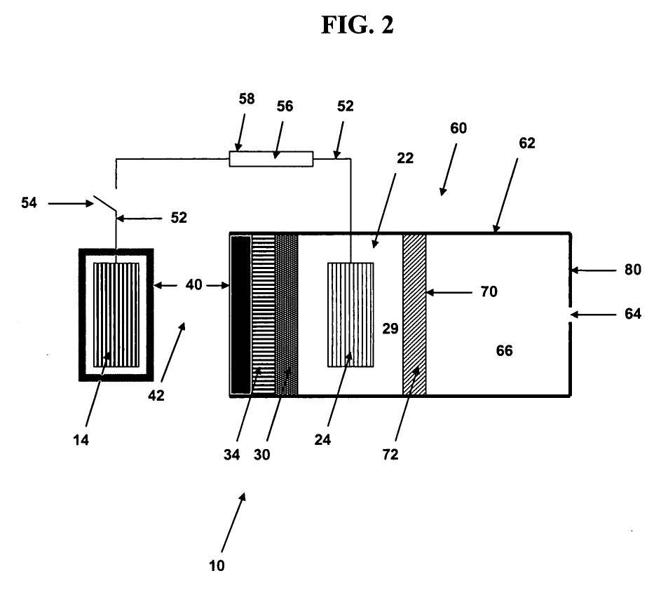

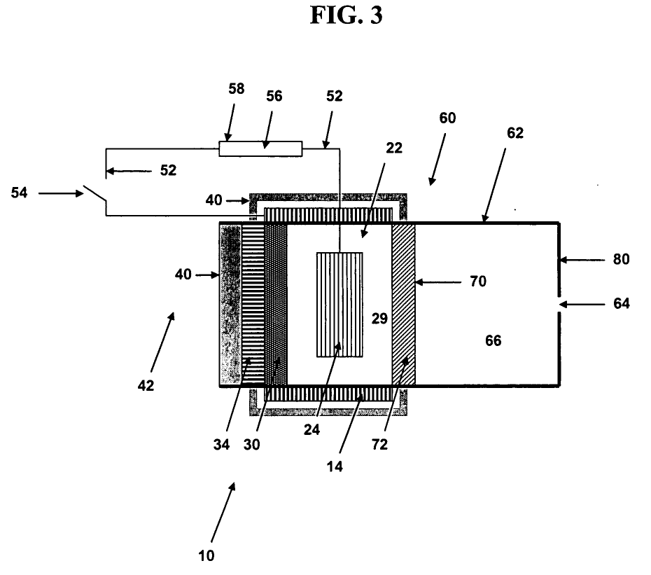

Alternatively, the first electrode 14 need not be positioned inside the device and can be positioned either entirely away from the housing (FIG. 2) or on the outside wall of the device (FIG. 3). In that case the ion exchange membrane 30 has more direct access to the body fluid and a porous separator 40 can be placed directly adjacent to the ion-exchange membrane 30 to prevent biofouling and to prevent unwanted species from contacting the membrane directly. This configuration will also eliminate trapping of any unwanted solid, liquid, or gaseous species in the auxiliary chamber and near the membrane 30.

Alternatively, while the use of the protective porous separator 40 is generally desirable for applications within the body, the separator is not required, especially in the case where necessary water or saline is self-contained in the auxiliary electrode compartment without any migration of water from external source 46. In that case the first half-cell 12 retracts or collapses around the auxiliary electrode on transfer of water from the first half-cell 12 to second half-cell 22 via electrosomosis. In such an embodiment, the first half-cell 12 can be exposed directly to fluid.

The housing 80 shown in FIG. 1 is generally an elongated cylindrical containing the first half-cell 12 and the second half-cell 22. The housing 80 may be constructed of metal, glass, natural and synthetic plastics, composites, or a combination thereof. The first half-cell 12 is positioned between the ion-exchange membrane 30 and the protective porous separator or protective gel 40, and is capable of containing water and electrolytic products that are controllably generated during the initiation of the current.

The second half-cell 22 is positioned between a displaceable member 70 and the first half-cell 12, and is capable of containing water 29 and electrolytic products that are controllably generated during operation of first half-cell 12.

A support member(s) 34 is configured proximate the ion-exchange membrane 30 and the first half-cell 12. The support member(s) 34 provide mechanical rigidity for the ion-exchange membrane 30 and allows water to transport through it. The support member 34 can be made of hard plastic, ceramic, glass, corrosion stable metal (e.g., titanium), or other like materials known to those with ordinary skilled in the art.

The fluid delivery device includes a fluid reservoir 66 having at least one exit aperture or port 64. The electro-osmotic cell 60 operates to steadily and consistently deliver fluid from the reservoir 66 until operations are halted. The displaceable member 70 is slideably associated within the device 60 so that, as the volume of fluid contained within second half-cell 22 increases, the displaceable member 70 is correspondingly maneuvered into the reservoir 62 to expel fluid out. For illustrative purposes, the displaceable member 70 positioned between the reservoir 62 and the second half-cell 22 is shown as comprising a piston 72, however, other configurations for the displaceable member known to those having ordinary skill in the art having the present disclosure before them are likewise contemplated for use, including and not limited to: a bladder, diaphragm, plunger, and bellows.

The reservoir 62 is capable of containing a fluid 66, such as a medicament, lubricant, fragrant fluid, chemical agent, or mixtures thereof, which is/are delivered via operation of the electro-osmotic delivery device 60. The term “fluid” is herein defined as a liquid, gel, paste, or other semi-solid state material that is capable of being delivered out of the reservoir 62.

In an effort to better understand the present invention, an exemplification incorporating materials capable of being utilized in the fluid delivery device is provided wherein the first electrode 14 is made of zinc and the second electrode 24 is made of silver chloride. In such a configuration, the following reactions take place wherein first electrode 14, e.g., zinc, is dissolved according to the equation:
Zn→Zn²⁺+2e⁻ (1)

Zinc ions thus formed are dissolved in water and migrate under the influence of the electric field. Sodium ions present in the electrolyte also migrate under the influence of the electric field and are expected to constitute the primary current carrying ion. These cations migrate through the ion-exchange membrane 30 towards the second electrode 24 in the second half-cell 22.

At the second electrode 24, silver chloride is reduced to metallic silver releasing chloride ions into solution according to the equation:
2AgCl+2e⁻→2Ag+2Cl⁻ (2)

Zinc ions and sodium ions react with chloride ions forming zinc chloride and sodium chloride according to the equations:
Zn²⁺+2Cl⁻→ZnCl₂ (3)
Na⁺+Cl⁻→NaCl (4)

In addition to the electrochemical formation of zinc chloride and sodium chloride according to the above equations, during passage of the cations through the membrane 30, water is entrained with the cations so that an additional amount of water is transported to second half-cell 22. This water transport is known in the art as electro-osmotic transport. Since the cationic membrane 30 is an exchange for cations, only cations can pass through membrane. Therefore, water may be transported through the membrane only in one direction from first half-cell 12 to second half-cell 22. Due to the continuous formation of sodium chloride and zinc chloride, the steady buildup of ion concentration in the second half-cell 22 induces further water transport through environmental osmosis. Thus, a steady state flux of water transport into the second half-cell 22 is established over a period of time by the combined electro-osmotic and osmotic effects. It should be noted that the osmotic flux is the result of the necessary concentration gradient. Therefore, the osmotic flux can be modified by virtue of modifying the electro-osmotic driving force. This is not possible with osmosis based devices and therefore, the delivery rate of an osmosis based device is not adjustable.

The formed zinc chloride, sodium chloride and water molecules increase the volume within second half-cell 22. The increased volume, in turn, generates pressure in the second half-cell 22 and imparts a force upon the displaceable member 70 and moves the member 70 laterally away from second half-cell 22, which controllably expels fluid from the reservoir 62. It will be understood that the above-identified device and process enables a controlled delivery of a fluid over an extended period of time at a relatively precise and accurate rate inasmuch as the water transported is proportional to the current, which in turn depends on the value of the resistor 58. Therefore, the fluid delivery rate is controlled by selection of the resistor 58 or controller output and not by the rate at which water is permitted to enter the device via convection action of protective porous separator 40.

Although today's electro-osmotic delivery device is effective in delivering fluid through electro-osmotic transport, the amount of time required to achieve a consistent fluid delivery rate can be quite long. During operation, an increase in the salt concentration within one of the half-cells, e.g., second half-cell, can be observed, which can adversely affect electro-osmotic cell operations by causing additional osmotic transport within the cell. The slow buildup of steady-state ion concentration translates into slow establishment of steady state flux at the start of the operation of the device. This additional transport slowly increases until steady-state concentrations are reached in both the half-cells.

The present invention incorporates a methodology directed to minimizing the effects associated with the slow startup phenomenon common in today's electro-osmotic delivery devices. In general, the present invention incorporates a variety of methods that can be utilized to achieve a faster delivery startup. A first method involves the electro-osmotic cell having a pre-configured concentration gradient so that one of the half-cells 12, 22 contains a higher concentrated solution than the other. A second method achieves a faster delivery startup by utilizing a controller to pass higher current between the two half-cells 12, 22 at the onset of the device operation.

With respect to the first method, the fluid delivery device 60 of the present invention includes a pre-configured concentration difference established within the cell 10 prior to operation of the device. That is, the first half-cell 12 and second half-cell 22 are pre-loaded with an electrolyte having a first initial ionic concentration and second initial ionic concentration that is greater than the first initial ionic concentration. After pre-loading the first 12 and second 22 half-cells, the activation switch 54 is actuated, whereupon an electrical circuit is completed to cause electrode reactions to take place at the electrodes 14, 24 and water to be extracted from the external environment 42, and, ultimately to be driven across the ion-exchange membrane 30 into the second half-cell 22. Thus, water from the external environment, such as a human body, diffuses through the protective porous separator 40 into the first half-cell 12. In the case where the separator 40 is not used, fluid will come in direct contact with the first electrode 14.

The presence of the greater steady-state ionic concentration in the second half-cell 22 enables achievement of a constant delivery rate of the fluid, e.g., medicament, quickly after initiation of the current flow within the device. That is, the effect of the variable rate, i.e., slow increase, of environmental osmosis initiated at the start of the fluid delivery device's operation prior to achieving a constant delivery rate can be minimized by the configuration of the present invention wherein unequal steady-state ionic concentrations are present within the two half-cells.

The constant delivery rate is maintained throughout the operation of the fluid delivery device, as the second ionic concentration will be maintained constant by the application of constant current, which, in turn, ensures that environmental osmosis is substantially constant; thus, increasing the reliability, predictability, and consistency of the delivery of fluid from the device.

The pre-established ion concentration in the second half-cell 22 determines the steady-state delivery rate—higher concentrations will result in higher delivery rates due to larger environmental osmosis—and can be determined prior to the operation of the device as follows. To determine the steady-state delivery rate, the fluid delivery device is operated normally with the same starting ionic concentration in both of the half-cells (without the pre-established concentration). The steady-state ionic concentration required for providing the pre-established concentration can be determined after the steady-state delivery rate is achieved.

A second approach to minimize the effects associated with the slow startup phenomenon utilizes a controller, e.g., resistor, to quickly achieve the steady-state ion concentration in the second half-cell 22. This is achieved by providing an initial current greater than the normal operating current between the two half-cells 12, 22 at the start of the device operation. The initial current can be provided by decreasing the electrical resistance between the electrodes 14, 24. The initial current can be determined by operating the fluid delivery device with a resistance value wherein the desired steady-state delivery rate is achieved and maintained. To achieve the desired steady-state delivery rate more quickly, the resistance value can be proportionally lowered to allow passage of a higher current between the electrodes 14, 24. This will result in achieving the steady-state delivery rate in an expeditious manner by quick establishment of steady-state concentration. Upon achieving the steady-state delivery rate, the resistance value can be increased to maintain the delivery rate.

While specific embodiments of the present invention have been illustrated and described numerous modifications come to mind without significantly departing from the spirit of the invention and the scope of protection is only limited by the scope of the accompanying claims.

What is claimed is:

1. A fluid delivery device, comprising:
an electrochemical cell including a first half-cell and a second half-cell;
a first electrode positioned within the first half-cell;
a second electrode positioned within the second half-cell;
a controller operably coupled to the first and second electrodes;
a means for decreasing the time to achieve a steady-state osmotic transfer;
an ion-exchange member positioned between the first half-cell and the second half-cell;
a first reservoir containing a fluid to be delivered; and,
a displaceable member positioned between the electrochemical cell and the reservoir, wherein movement of the displaceable member facilitates delivery of the fluid from the reservoir.

2. The fluid delivery device of claim 1, further comprising:
at least one protective porous separator or gel that is permeable to H₂O molecules or saline, wherein the at least one protective separator or gel is positioned such that when the fluid delivery device being implanted within a body, at least one the electrodes or the ion-exchange membrane is not directly exposed to body tissue or body fluid.

3. The fluid delivery device of claim 2, wherein the protective porous separator is a second reservoir operably connected to the first half-cell, the second reservoir being a water or saline reservoir.

4. The fluid delivery device of claim 2, wherein the protective porous separator is a gel.

5. The fluid delivery device of claim 1, further comprising:
a housing wherein the first half-cell or the second half-cell being located outside of the housing.

6. The fluid delivery device of claim 1, further comprising:
a housing wherein the second half-cell and the ion-exchange member being located within the housing.

7. The fluid delivery device of claim 1, wherein the ion-exchange member is a cationic exchange membrane.

8. The fluid delivery device of claim 7, wherein the first electrode and the second electrode comprise zinc and silver chloride, respectively.

9. The fluid delivery device of claim 1, wherein the ion-exchange member is an anionic exchange membrane.

10. The fluid delivery device of claim 9, wherein the first electrode and the second electrode comprise silver chloride and zinc, respectively.

11. The fluid delivery device of claim 1, further comprising:
an electrolyte in electrical communication with the first electrode and the second electrode.

12. The electro-osmotic cell according to claim 11, wherein the electrolyte comprises a bodily fluid.

13. The fluid delivery device of claim 1, wherein the fluid contained in the first reservoir is a medicament.

14. The fluid delivery device of claim 1, wherein the displaceable member comprises a piston, bladder, bellows, diaphragm, plunger, or a combination thereof.

15. The fluid delivery device of claim 1 wherein the means for decreasing the time to achieve a steady-state osmotic transfer includes:
the first half-cell having a first ionic concentration; and,
the second hall-cell having a pre-established ionic concentration greater than the concentration of the first half-cell,
wherein environmental osmosis between the first half-cell and the second half-cell is quickly established.

16. The fluid delivery device of claim 1 wherein the means for decreasing the time to achieve a steady-state osmotic transfer includes a means for varying the current between the first and second half-cells.

17. A method for decreasing the time to achieve constant fluid delivery rate in a fluid delivery device, the method comprising the steps of:
providing an electrochemical cell adjacent a fluid reservoir, the electrochemical cell including a first half-cell and a second half-cell, wherein the electro-osmotic cell facilitates delivery of a fluid from the fluid reservoir;
pre-loading the first half-cell with an electrolyte having a first ionic concentration; and,
pre-loading the second half-cell with an electrolyte having a second ionic concentration that is greater than the first ionic concentration of the first half-cell, wherein environmental osmosis between the first half-cell and the second half-cell is quickly established.

18. A method for decreasing the time to achieve a constant fluid delivery rate in a fluid delivery device, the method comprising the steps of:
providing an electrochemical cell adjacent a fluid reservoir, the electrochemical cell including a first half-cell and a second half-cell, wherein the electro-osmotic cell facilitates delivery of a fluid from the fluid reservoir;
generating a first electrical current between the first half-cell and the second half-cell to achieve a steady-state osmotic transfer between the first half-cell and the second half-cell; and,
reducing the first electrical current in response to achieving a desired fluid delivery rate such that the fluid delivery rate is maintained constant.
